(19) United States
(12) Patent Application Publication
(10) Pub. No.: US 2022/0372169 A1
(43) Pub. Date: Nov. 24, 2022

(54) INHIBITION OF THE VE-PTP PHOSPHATASE PROTECTS THE KIDNEY FROM ISCHEMIA-REPERFUSION INJURY

(71) Applicants: Northwestern University, Chicago, IL (US); Mannin Research Inc., Toronto (CA)

(72) Inventors: Susan E. QUAGGIN, Chicago, IL (US); Michael C. RYCZKO, Toronto (CA); Amy S. RIPKA, Reading, MA (US)

(21) Appl. No.: 17/774,755

(22) PCT Filed: Oct. 30, 2020
(86) PCT No.: PCT/US2020/058245

§ 371 Date: May 5, 2022

Related U.S. Application Data
(60) Provisional application No. 62/931,686, filed on Nov. 6, 2019.

(51) Int. Cl. C07K 16/40; A61P 13/12

(52) U.S. Cl. CPC C07K 16/40; A61P 13/12; C07K 2317/76; A61K 2039/505

(57) ABSTRACT

This disclosure relates to inhibition of the VE-PTP phosphatase to protect the kidney from ischemia-reperfusion injury. This disclosure also relates to conditional knockout of VE-PTP to protect or improve the renal function in ischemia-reperfusion injury. This disclosure identifies VE-PTP as a promising therapeutic target for renal protection in ischemia-reperfusion injury and proposes using small molecule VE-PTP inhibitor to bestow protection in the context of acute kidney injury.

### FIELD OF THE INVENTION

The invention relates to acute kidney disease and any other kidney disease, and more particularly to use of VE-PTP inhibition and Tie2 activation for protection of renal function and alleviation of acute kidney injury symptoms.

### BACKGROUND OF THE INVENTION

The endothelial angiopoietin (ANG)-Tie2 signaling pathway is required for vascular development, maintenance of endothelial stability, integrity and homeostasis. Dysregulation of Ang-Tie2 pathway has been implicated in diseases including venous malformation, glaucoma, vascular leakage, diabetic nephropathy, sepsis, ischemia-reperfusion injury, and acute kidney injury (AKI). Tie2 (TEK) receptor tyrosine kinase expression is heavily enriched in vascular endothelium. The endothelial-specific phosphatase VE-PTP, encoded by gene PTPRB, is a crucial negative regulator of Tie2 phosphorylation and activation status. Inhibition of VE-PTP is a promising therapeutic target for diabetic kidney injury in mice, but its role in acute kidney injury has hitherto not been studied. This invention relates to inhibition of VE-PTP to protect the kidney from acute kidney injury due to ischemia-reperfusion (IR) injury, and to slow and/or reduce renal dysfunction in patients.

### SUMMARY OF THE DISCLOSURE

Some embodiments of the invention include a method of treating a patient with acute kidney injury or disease by administering a pharmaceutical composition comprising agents capable of TIE2 receptor activation, such as VE-PTP inhibitors or Angiopoietin recombinant or chimeric proteins.

The invention also includes a pharmaceutical composition for subcutaneous delivery and controlled sustained release comprising an effective dosage amount of Tie2 receptor activating agent such as VE-PTP inhibitors or Angiopoietin recombinant or chimeric proteins for treatment of acute kidney injury.

In an embodiment of the present invention, inhibition of VE-PTP protects the kidney from acute kidney injury due to ischemia-reperfusion injury. The invention also includes a pharmaceutical composition comprising a pharmaceutically active amount of the TIE2 receptor activating agent, i.e. VE-PTP inhibitor, formulated for subcutaneous extended release delivery for treatment of acute kidney injury.

The invention also includes a pharmaceutical composition comprising a pharmaceutically active amount of the TIE2 receptor activating agent, i.e. VE-PTP inhibitor, formulated for subcutaneous extended release delivery for treatment of acute kidney injury.

The additional embodiments also constitute part of the disclosure:

1. A method of treating a patient with acute or chronic kidney injury or disease (which affect kidney function), or ischemia-reperfusion injury (for example, kidney, lung), in a subject in need thereof, the method comprising administering to the subject a pharmaceutical composition comprising one or more agents capable of TIE2 receptor activation, such as VE-PTP inhibitors or Angiopoietin recombinant or Angiopoietin chimeric proteins to the subject.

2. A pharmaceutical composition for subcutaneous delivery and controlled sustained release comprising an effective dosage amount of one or more Tie2 receptor activating agent such as VE-PTP inhibitors, Angiopoietin recombinant or Angiopoietin chimeric proteins for treatment of acute kidney injury or disease, or ischemia-reperfusion injury, optionally wherein the composition is an hydrogel.

3. A method of protecting the kidney from acute kidney injury or disease due to ischemia-reperfusion injury, or ischemia-reperfusion injury in a subject in need thereof, the method comprising administration of an inhibitor of VE-PTP to the subject.

4. A pharmaceutical composition comprising a pharmaceutically active amount of the TIE2 receptor activating agent, i.e. VE-PTP inhibitor, formulated for subcutaneous extended release delivery for treatment of acute kidney injury or disease, or ischemia-reperfusion injury.

5. A method of reducing/preventing activation of the renal endothelium, reducing/preventing a pro-inflammatory endothelial state, reducing macrophage accumulation, reducing fibrotic response after injury (e.g., renal IR injury), and/or downregulation/upregulation of acute stress response genes (e.g. VCAM1(down), E-selectin (down), Angpt2 (down), Entpd1 (upregulation), Cyr61 (down), endothelial activation gene signatures) (in injured tissue), down-regulation of HIF2-alpha, in a subject in need thereof, the method comprising administering to the subject a pharmaceutical composition comprising one or more agents capable of TIE2 receptor activation, such as VE-PTP inhibitors or Angiopoietin recombinant or Angiopoietin chimeric proteins to the subject.

6. A method for protection and/or improvement of renal function and alleviation of acute kidney injury symptoms in a subject in need thereof, the method comprising administering to the subject a pharmaceutical composition comprising one or more agents capable of TIE2 receptor activation, such as VE-PTP inhibitors or Angiopoietin recombinant or Angiopoietin chimeric proteins to the subject.

7. The method or treatment of any one of embodiments 1 through 6, wherein the one or more agents capable of TIE2 receptor activation, such as VE-PTP inhibitors or Angiopoietin recombinant or Angiopoietin chimeric proteins are administered to the subject prior to the infliction of acute kidney injury or disease, or ischemia-reperfusion injury; during infliction of acute kidney injury or disease, or ischemia-reperfusion injury; and/or after infliction of acute kidney injury or disease, or ischemia-reperfusion injury.

8. The method or treatment of any one of embodiments 1 through 7, wherein ischemia reperfusion injury occurs in the setting of acute coronary syndrome, acute kidney injury, intestinal ischemia and reperfusion, stroke, sickle cell disease, sleep apnea, major surgery, or solid organ transplantation.

9. The method or treatment of any one of embodiments 1 through 8, wherein inhibition of VE-PTP is used in combination with anti-inflammatory and/or anti-oxidant therapy/ies.

10. The method or treatment of any one of embodiments 1 through 9, wherein the agent(s) activate(s) the Tie2 receptor either directly or by inhibiting its negative regulator VE-PTP, optionally, are selected from the agents described in paragraph [020] of the application.

11. The method or treatment of any one of embodiments 1 through 10, wherein renal/kidney function is measured by any method know by one of ordinary skill in the art (albumin to creatinine ratio, albuminuria, serum urea, inulin clearance, radioisotopic methods, radiocontrasting agents, Cystatin C, and/or glomerular filtration rate and staging of kidney disease).

12. The method or treatment of any one of embodiments 1 through 11, wherein signs and symptoms of kidney injury/failure include too little urine leaving the body, blood in urine, Swelling in legs, ankles, and/or around the eyes, Fatigue or tiredness, Shortness of breath, Seizures or coma in severe cases, confusion, nausea, chest pain or pressure, high blood pressure, dehydradation, drowsiness, hemorrhage, fever, rash, bloody diarrhea, severe vomiting, abdominal pain, pale skin, edema, and/or detectable abdominal mass.

13. The method or treatment of any one of embodiments 1 through 12, wherein kidney injury is caused by infection, dehydration, recent surgery, trauma, exposure to heavy metals or toxic solvents, a condition that obstructs blood flow (e.g., cardia arrest), medications, kidney stones, blood vessel abnormalities that affect blood flow to/from/within the kidney, glomerulonephritis, lupus, blockage in the ureters, low blood pressure, bleeding too much, severe diarrhea, heart disease or heart attack, liver failure, non-steroidal anti-inflammatory drugs (e.g., aspirin, ibuprofen, naproxen), serious burns, severe allergic reaction, blood cloths in or around the kidneys, chemotherapy, antibiotics, contrast dyes used during CT scans, MRI scans, and other imaging tests, alcohol abuse, drug abuse, cancer, enlarged prostate, diabetes, virus infections (e.g., coronavirus).

14. The method or treatment of any one of embodiments 1 through 13, wherein the treatment is combined with hemodialysis, peritoneal dialysis, medicines to control the amounts of vitamins and minerals (e.g., potassium, calcium) in the blood (e.g., calcium, glucose or sodium polystyrene sulfonate (Kionex), treatments to keep the right amount of fluid in the blood (e.g., IV fluids, diuretics), diet, kidney transplant, steroids, acthar, rituximab, cyclophosphamide, mycophenalate mofetil, ACE inhibitors, angiotensin II receptor blockers, cyclosporine, tracrolimus, sirolimus, liposorber LA-15, and combinations thereof.

15. A VE-PTP knockout mice produced by a method that comprises the steps of FIG. 1.

16. A VE-PTP knockout mice wherein a bitransgenic doxycycline-inducible system (Veptp^{flox/flox}, Rosa26-rtTA^{+/+}, tetO-Cre^{Tg/+}) is used to knockout the VE-PTP gene from the vasculature of mice at postnatal day 0 (VE-PTPiKO).

17. Any embodiment as described in the Figures and Examples of this application.

### BRIEF DESCRIPTION OF THE FIGURES

FIG. 1 shows the strategy used to generate inducible VE-PTP knockout mice model. A bitransgenic doxycycline-inducible system (Veptpflox/flox, Rosa26-rtTA+/+, tetO-CreTg/+) was used to knockout the VE-PTP gene from the vasculature of mice at postnatal day 0 (VE-PTPiKO).

FIG. 2 shows VE-PTP is upregulated in kidney with ischemia-reperfusion injury. (A) Adult male VE-PTPiKO and littermate control mice underwent 20 minutes of bilateral renal ischemia reperfusion injury (IR) or sham surgery. Western blot analysis in whole-kidney extracts showed elevated VE-PTP level after 1 day and 7 days of reperfusion. Error bars shown as s.e.m. and significance determined by one-way ANOVA with Tukey correction for multiple comparisons. (B) Systematic over expression of HIF2-alpha resulted in elevated kidney VE-PTP levels. Values are means±SD. ****P<0.0001; **P<0.01; *P<0.05; ns, P>0.05.

FIG. 3 shows targeting VE-PTP improves ischemic renal function in young mice. A) Baseline creatinine level in wild type (control) and VE-PTP induced knockout (VE-PTPiKO) male mice at 3 months. (B) Creatinine level of 3-month-old control and VE-PTPiKO mice at indicated timepoints after IR (two-way ANOVA). (C) Creatinine level of 1-year-old control and VE-PTPiKO mice at 24 hours after IR. Serum creatinine was measured by HPLC method. (D) Western blot and immunoprecipitation (IP) analysis of lung tissue showed deletion of VE-PTP increased TIE2 phosphorylation. (E) mRNA was extracted from mice kidney 7 days after IR. Values are means±SEMs. ***, P<0.001; *, P<0.05; ns, P>0.05.

FIG. 4 shows controlled VE-PTP inhibitor release following hydrogel depot injection increases Tie2 phosphorylation. Western blot and immunoprecipitation (IP) analysis of lung tissue showed increased TIE2 phosphorylation after subcutaneous injection of hydrogel containing 8 mg/ml VE-PTP inhibitor at a dosage of 32 ul per gram body weight (A), with negligible effect on VEGFR2 phosphorylation (B). Experiments were repeated at least once.

FIG. 5 shows genetic inactivation of VE-PTP in the kidney reduces macrophage accumulation and fibrotic response after renal IR injury. (A) Kidneys harvested on day 7 were stained with CD68 to determine macrophage accumulation Immunohistochemistry identified less immune inflammatory CD68 positive cells in the outer medulla area of the kidney in the VE-PTP knockout mice. (B) Expression of pro-fibrotic genes such as Connective Tissue Growth Factor (CTGF), Fibronectin (Fn1) and Snail1 was significantly reduced on day 7 in the VE-PTP deficient kidneys compared to controls.

FIG. 6 shows genetic inactivation of VE-PTP in kidney results in less pro-inflammatory endothelial state after bilateral renal IR injury. (A) Transcriptome analysis revealed downregulation of several marker genes for endothelial activation (VCAM1, E-Selectin and Angpt2), upregulation of protective gene Ectonucleoside triphosphate diphosphohydrolase-1 (Entpd1), and downregulation of Cysteine-rich protein 61 (Cyr61), an early biomarker of AKI. For RNA analysis, bulk RNAseq was performed with total RNA extracted from whole kidney. Sequences were aligned to the Mus musculus genome (mm10) using STAR, with normalization and differential expression determined using DESeq2. (B) Change in gene expression was confirmed using qPCR normalized to GAPDH in three month old VE-PTP knockout males and littermate control mice that underwent bilateral renal IR injury or sham surgery.

### DESCRIPTION

Except as otherwise expressly provided herein, each of the following terms shall have the meaning set forth below. Additional definitions are set forth throughout the application. Unless defined otherwise, all technical and scientific terms used herein have the meaning as commonly understood by one of ordinary skill in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this application.

Units, prefixes, and symbols are denoted in their Systeme International de Unites (SI) accepted form. Numeric ranges are inclusive of the numbers defining the range. The disclosure provided herein are not limitations of the various aspects of the application, which may be by reference to the specification as a whole.

The articles “a” or “an” refer to “one or more” of any recited or enumerated component.

The terms “about” or “comprising essentially of” refer to a value or composition that is within an acceptable error range for certain value or composition as determined by one of ordinary skill in the art, which will depend in part on how the value or composition is measured or determined, i.e., the limitations of the measurement system. For example, “about” or “comprising essentially of” may mean within 1 or more than 1 standard deviation per the practice in the art. Alternatively, “about” or “comprising essentially of” may mean a range of up to 10% (i.e., ±10%). For example, about 3 mg may include any number between 2.7 mg and 3.3 mg (for 10%). With respect to biological systems or processes, the terms may mean up to an order of magnitude or up to 5-fold of a value. When certain values or compositions are provided in the application and claims, unless otherwise stated, the meaning of “about” or “comprising essentially of” include an acceptable error range for that value or composition. Any concentration range, percentage range, ratio range, or integer range includes the value of any integer within the recited range and, when appropriate, fractions thereof (such as one-tenth and one-hundredth of an integer), unless otherwise indicated.

The term “and/or” refer to each of the two specified features or components with or without the other. Thus, the term “and/or” as used in a phrase such as “A and/or B” herein is intended to include “A and B,” “A or B,” “A” (alone), and “B” (alone). Similarly, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” are used merely by way of example, without limitation intended, and not be construed as referring only those items explicitly enumerated in the specification.

The terms “or more”, “at least”, “more than”, and the like, e.g., “at least one” include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between. The term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between

Throughout the specification the word “comprising,” or variations such as “comprises” or “comprising,” is understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

The term “activation,” “activated,” or the like refers to the state of a cell, including and not be limited to an endothelial cell, that has been sufficiently stimulated to induce detectable cellular proliferation.

The terms “administration,” “Administering” or the like refer to physical introduction of an agent to a subject, using any of the various methods and delivery systems known to those skilled in the art. Exemplary routes of administration for the drugs or agents prepared by the methods disclosed herein include intravenous (i.v. or IV), intramuscular, subcutaneous, intraperitoneal, spinal or other parenteral routes of administration, for example by injection or infusion. Parenteral route of administration refer to modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the agents prepared by the present methods are administered via injection or infusion. Non-parenteral routes include a topical, epidermal or mucosal route of administration, for example, intranasally, vaginally, rectally, sublingually or topically. Administering may also be once, twice, or a plurality of times over one or more extended periods. Where one or more therapeutic agents (e.g., cells) are administered, the administration may be done concomitantly or sequentially. Sequential administration comprises administration of one agent only after administration of the other agent or agents has been completed.

A “therapeutically effective amount,” “therapeutically effective dosage,” or the like refers to an amount of the agent that are produced by the present methods and that, when used alone or in combination with another therapeutic agent, protects or treats a subject against the onset of a disease or promotes disease regression as evidenced by a decrease in severity of disease symptoms, an increase in frequency and duration of disease symptom-free periods, and/or prevention of impairment or disability due to disease affliction. The ability to promote disease regression may be evaluated using a variety of methods known to the skilled practitioner, such as in subjects during clinical trials, in animal model systems predictive of efficacy in humans, or by assaying the activity of the agent in in vitro assays. There are many mouse models of renal ischemia reperfusion injury in addition to the model described in this application. See, e.g., Guan, Y., Nakano, D., Zhang, Y. et al. A mouse model of renal fibrosis to overcome the technical variability in ischaemia/reperfusion injury among operators. Sci Rep 9, 10435 (2019) doi:10.1038/s41598-019-46994-z and there are also commercially available customized preclinical services to study the effects of new agents in kidney ischemia/reperfusion injury.

The terms cell “proliferation,” “proliferating” or the like refer to the ability of cells to grow in numbers through cell division. In some embodiments, proliferation may be measured by staining cells with carboxyfluorescein succinimidyl ester (CFSE). Cell proliferation may occur in vitro, e.g., during endothelial cell culture, or in vivo. The cell proliferation may be measured or determined by the methods described herein or known in the field. For example, cell proliferation may be measured or determined by viable cell density (VCD) or total viable cell (TVC). VCD or TVC may be theoretical (an aliquot or sample is removed from a culture at certain timepoint to determine the cell number, then the cell number multiples with the culture volume at the beginning of the study) or actual (an aliquot or sample is removed from a culture at certain timepoint to determine the cell number, then the cell number multiples with the actual culture volume at the certain timepoint).

A “patient” as used herein includes any human who is afflicted with a disease or disorder, including kidney disease. The terms “subject” and “patient” are used interchangeably herein. In one embodiment, the patient is a human. In one embodiment, the patient is an animal.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

“Treatment” or “treating” of a subject refers to any type of intervention or process performed on, or the administration of one or more agents or drugs prepared by the present application to, the subject with the objective of reversing, alleviating, ameliorating, inhibiting, slowing down or preventing the onset, progression, development, severity or recurrence of a symptom, complication or condition, or biochemical indicia associated with a disease. In one aspect, “treatment” or “treating” includes a partial remission. In another aspect, “treatment” or “treating” includes a complete remission.

The term “ischemia reperfusion” refers to a pathological condition due to an initial restriction of blood supply to an organ followed by the subsequent restoration of perfusion and concomitant reoxygenation. Ischemia reperfusion injury (IRI) contributes to morbidity and mortality in a wide range of pathologies like acute coronary syndrome, acute kidney injury, intestinal ischemia and reperfusion, stroke, sickle cell disease, sleep apnea, and solid organ transplantation (e.g., kidney transplantation).

Various aspects of the application are described in further detail in the following subsections.

In one embodiment, the disclosure provides a method of treating a patient with acute kidney injury or disease by administering a pharmaceutical composition comprising one or more agents capable of TIE2 receptor activation, such as VE-PTP inhibitors or Angiopoietin recombinant or chimeric proteins.

In one embodiment, the agents activate the Tie2 receptor either directly or by inhibiting its negative regulator VE-PTP. Recombinant or chimeric proteins of Angiopoietin-1 such as BowAng1 and COMP-Ang1 activate Tie2 directly. See, e.g. Davis, S. et al. Angiopoietins have distinct modular domains essential for receptor binding, dimerization and superclustering. Nat Struct Biol. 10(1):38-44 (2003) doi:10.1038/nsb880 and Oh, N. et al. A Designed Angiopoietin-1 Variant, Dimeric CMP-Ang1 Activates Tie2 and Stimulates Angiogenesis and Vascular Stabilization in N-glycan Dependent Manner. Sci Rep. 5, 15291 (2015) doi:10.1038/srep15291 Alternatively, there are also synthetic mimetic ligands and modulators that activate Tie2 including Vasculotide, TSL1 and AXT107. See, e.g. Dekker, N. A. et al. Vasculotide, an angiopoietin-1 mimetic, reduces pulmonary vascular leakage and preserves microcirculatory perfusion during cardiopulmonary bypass in rats. Br J Anaesth. 121(5) (2018) doi:10.1016/j.bja.2018.05.049, Issa, E. et al. Development of an Orthogonal Tie2 Ligand Resistant to Inhibition by Ang2. Mol Pharm. 4; 15(9) (2018) doi:10.1021/acs.molpharmaceut.8b00409 and Mirando, A. C. et al. A collagen IV-derived peptide disrupts α5β1 integrin and potentiates Ang2/Tie2 signaling. JCI Insight. 21; 4(4) (2019) doi:10.1172/jci.insight.122043. There are also different types of antibodies that act as Tie2 activators. These include the Angiopoietin-2—Binding and Tie2-Activating Antibody (ABTAA), anti-Tie2 receptor agonistic antibody, as well as antibodies targeting the extracellular domain of VE-PTP. See, e.g. Kim, J. et al. Tie2 activation promotes choriocapillary regeneration for alleviating neovascular age-related macular degeneration. Sci Adv. 13; 5(2) (2019) doi:10.1126/sciadv.aau6732, Hwang, B. et al. Stimulation of angiogenesis and survival of endothelial cells by human monoclonal Tie2 receptor antibody. Biomaterials 51:119-128 (2015) doi:10.1016/j.biomaterials.2015.01.062, and Frye, M. et al. Interfering with VE-PTP stabilizes endothelial junctions in vivo via Tie-2 in the absence of VE-cadherin. J Exp Med 14; 212(13) (2015) doi:10.1084/jem.20150718. There are also small molecule VE-PTP inhibitors that act as activators of Tie2, see, e.g. Campochiaro, P. A., Enhanced Benefit in Diabetic Macular Edema from AKB-9778 Tie2 Activation Combined with Vascular Endothelial Growth Factor Suppression. Ophthalmology. 123(8):1722-1730 (2016) doi:10.1016/j.ophtha.2016.04.025.

In one embodiment, the disclosure provides a pharmaceutical composition for subcutaneous delivery and controlled sustained release comprising an effective dosage amount of Tie2 receptor activating agent such as VE-PTP inhibitors or Angiopoietin recombinant or chimeric proteins for treatment of acute kidney injury. In one embodiment, an injectable hydrogel is used to deliver the VE-PTP inhibitor and is based on hydrazone cross-linked poly(oligo(ethylene glycol) methacrylate) or POEGMA. This two component system—aldehyde and hydrazide, upon mixing rapidly formed a crosslinked biodegradable hydrogel. 8% POEGMA in PBS can be used to encapsulate VE-PTP small molecule inhibitors for extended release. VE-PTP inhibitor solubility in POEGMA was as high as 25 mg/mL.

In an embodiment of the present disclosure, inhibition of VE-PTP protects the kidney from acute kidney injury due to ischemia-reperfusion injury.

In another embodiment, the disclosure provides a pharmaceutical composition comprising a pharmaceutically active amount of the TIE2 receptor activating agent, i.e. VE-PTP inhibitor, formulated for subcutaneous extended release delivery for treatment of acute kidney injury.

In one embodiment, ischemia reperfusion injury occurs in the setting of acute coronary syndrome, acute kidney injury, intestinal ischemia and reperfusion, stroke, sickle cell disease, sleep apnea, major surgery, or solid organ transplantation.

In one embodiment, inhibition of VE-PTP is used in combination with anti-inflammatory and anti-oxidant therapies.

### EXAMPLES

### Example 1

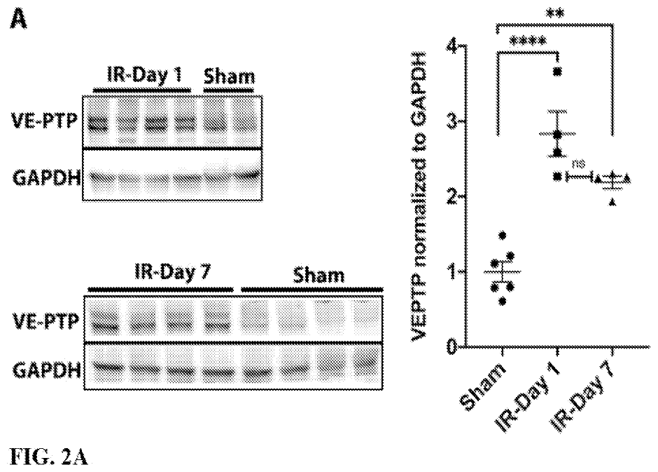

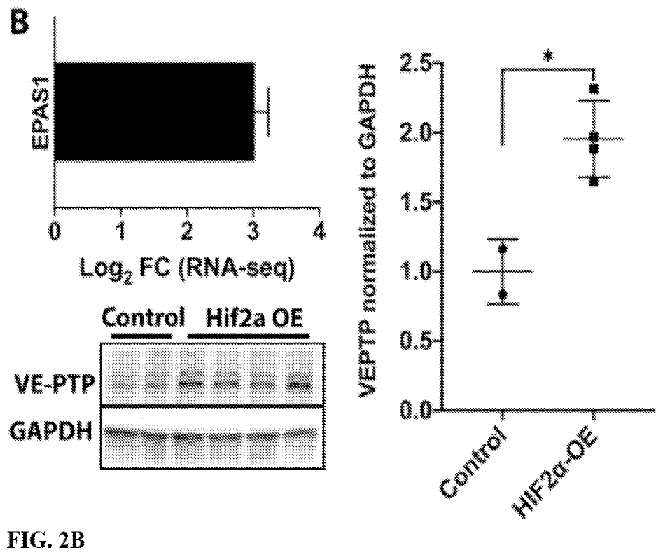

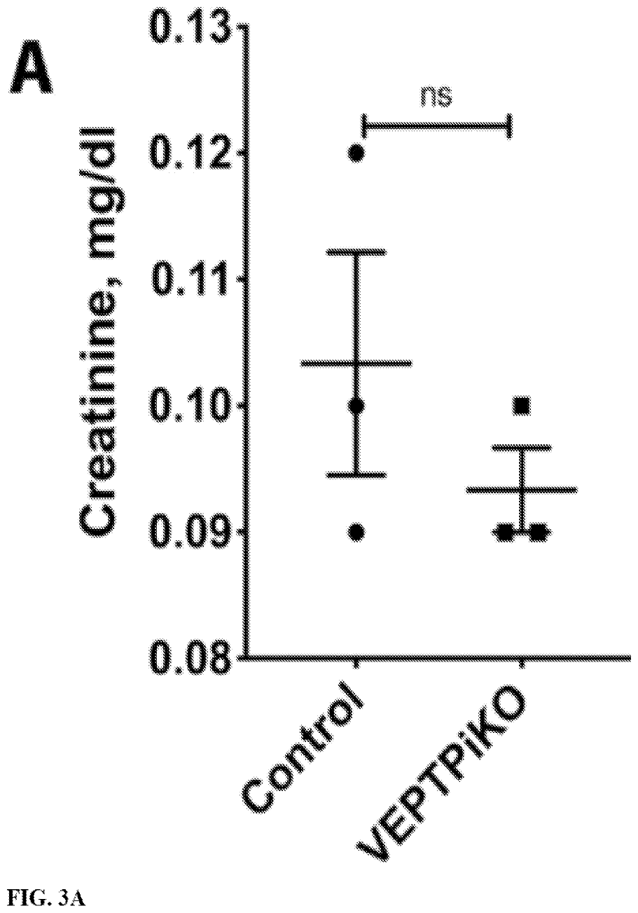

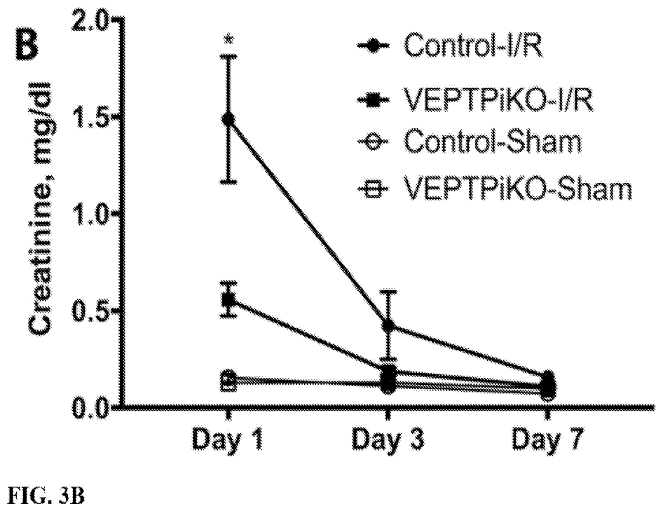

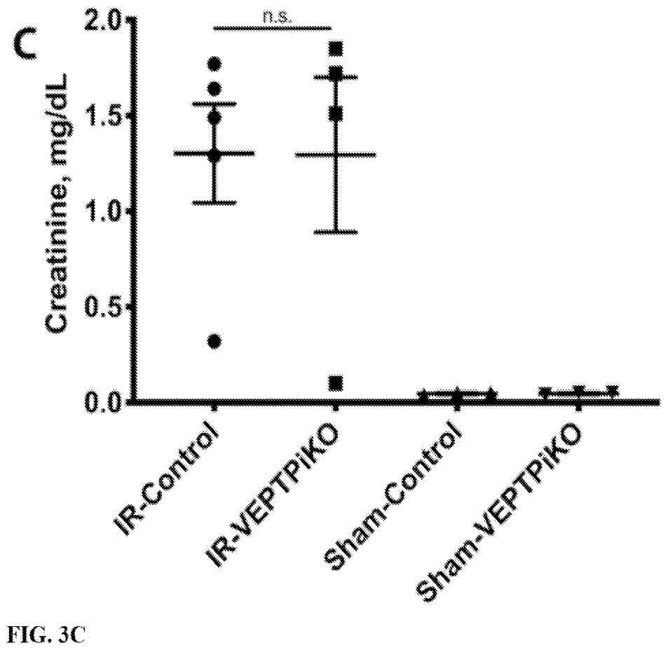

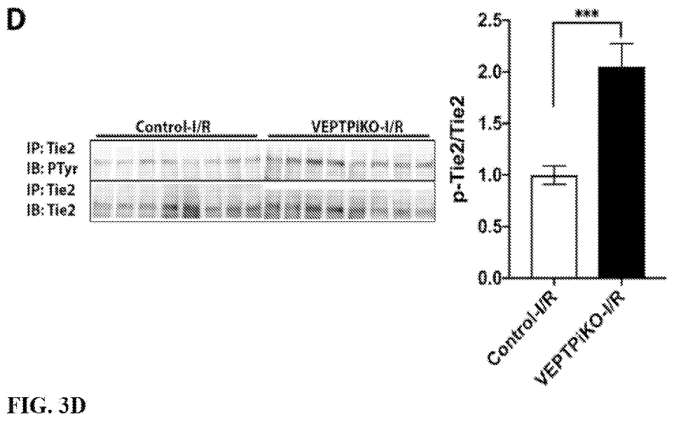

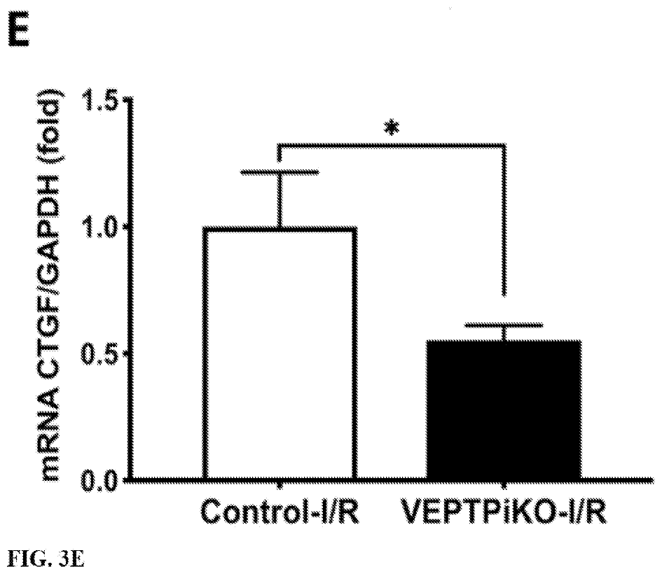

VE-PTP protein level is upregulated in kidneys post ischemia-reperfusion injury (FIG. 2A). Systemic transgenic overexpression of HIF2-alpha, confirmed by upregulation of Endothelial PAS domain-containing protein 1 (EPAS1), also results in elevated kidney VE-PTP levels (FIG. 2B). To determine renal health function serum creatinine was measured. The baseline Creatinine level in wild type control and VE-PTPiKO mice was in the same range (FIG. 3A). While serum Creatinine was elevated 1 day post-IR in control mice, this increase did not occur in VE-PTPiKO mice (FIG. 3B). This effect appeared to be age dependent (FIG. 3C).

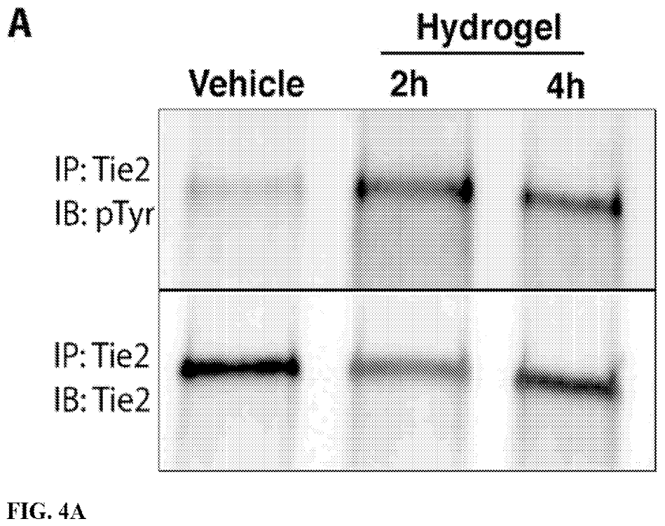

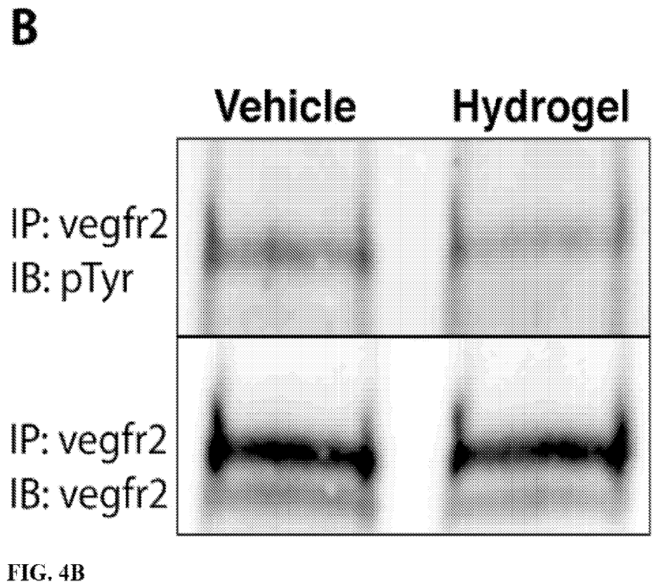

After IR injury, increase in pro-fibrotic factor and FOXO1 target gene CTGF was observed in control compared to VE-PTPiKO mice (FIG. 3E), illustrating the protective effect on the kidney associated with VE-PTP deficiency. Genetic deletion of VE-PTP robustly enhanced Tie2 phosphorylation and activation in vasculature of lung and kidney tissue in vivo (FIG. 3D). Pharmacological inhibition of VE-PTP, through subcutaneous injection of hydrogel for sustained release, also robustly enhanced Tie2 phosphorylation and activation in vasculature of lung tissue in vivo (FIG. 4A), with negligible effect on phosphorylation and activation of VEGFR2 (FIG. 4B).

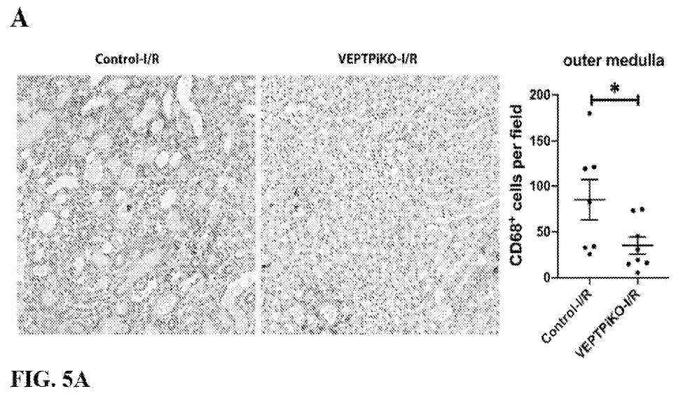

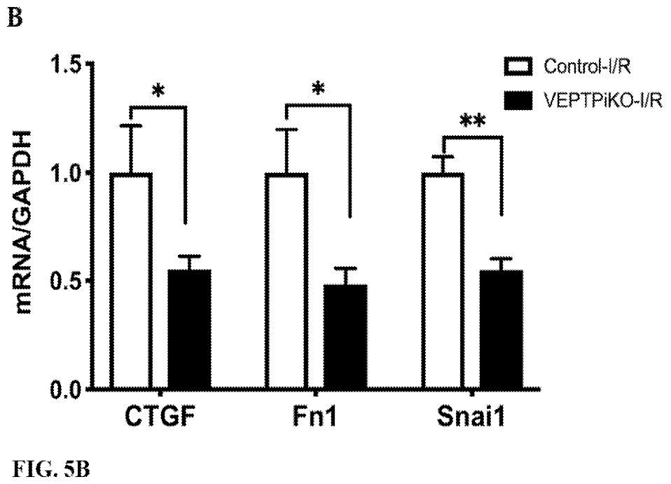

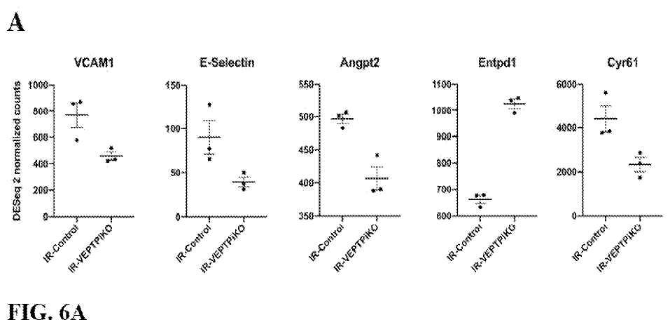

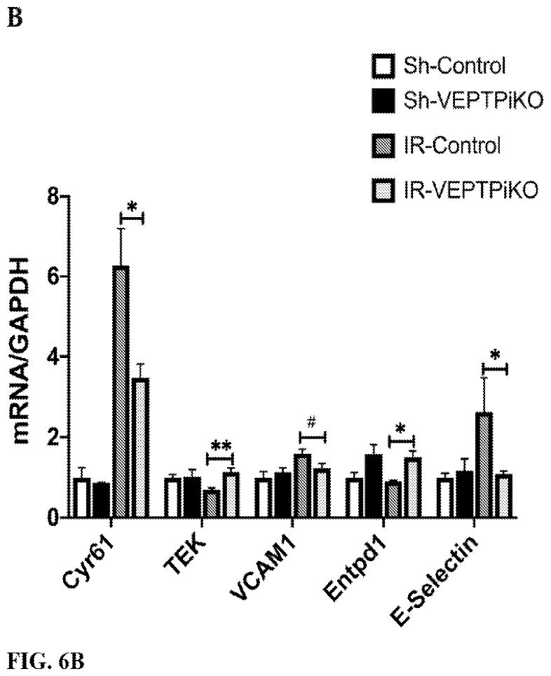

Loss of VE-PTP reduced macrophage accumulation and fibrotic response after renal ischemia reperfusion (IR) injury. Macrophage lineage marker CD68 was used to determine the extent of immune cell infiltration in the outer renal medulla. Following IR injury kidneys from WT control mice showed macrophage infiltration and intrarenal localization that was clearly detectable by day 3 after the insult (FIG. 5A). Genetic deletion of VE-PTP in the kidney decreased expression of pro-fibrotic genes in IR injury (FIG. 5B). Transcriptional profiling revealed that loss of VE-PTP in IR injury resulted in less activated renal endothelium, reduced pro-inflammatory endothelial state, and downregulation of acute stress response gene signature (FIG. 6).

1. A method of treating a patient with acute or chronic kidney injury or disease (which affect kidney function), or ischemia-reperfusion injury (for example, kidney, lung), in a subject in need thereof, the method comprising administering to the subject a pharmaceutical composition comprising one or more agents capable of TIE2 receptor activation, such as VE-PTP inhibitors or Angiopoietin recombinant or Angiopoietin chimeric proteins to the subject.

2. A pharmaceutical composition for subcutaneous delivery and controlled sustained release comprising an effective dosage amount of one or more Tie2 receptor activating agent such as VE-PTP inhibitors, Angiopoietin recombinant or Angiopoietin chimeric proteins for treatment of acute kidney injury or disease, or ischemia-reperfusion injury, optionally wherein the composition is an hydrogel.

3. A method of protecting the kidney from acute kidney injury or disease due to ischemia-reperfusion injury, or ischemia-reperfusion injury in a subject in need thereof, the method comprising administration of an inhibitor of VE-PTP to the subject.

4. A pharmaceutical composition comprising a pharmaceutically active amount of the TIE2 receptor activating agent, i.e. VE-PTP inhibitor, formulated for subcutaneous extended release delivery for treatment of acute kidney injury or disease, or ischemia-reperfusion injury.

5. A method of reducing/preventing activation of the renal endothelium, reducing/preventing a pro-inflammatory endothelial state, reducing macrophage accumulation, reducing fibrotic response after injury (e.g., renal IR injury), and/or downregulation/upregulation of acute stress response genes (e.g. VCAM1(down), E-selectin (down), Angpt2 (down), Entpd1 (upregulation), Cyr61 (down), endothelial activation gene signatures) (in injured tissue), down-regulation of HIF2-alpha, in a subject in need thereof, the method comprising administering to the subject a pharmaceutical composition comprising one or more agents capable of TIE2 receptor activation, such as VE-PTP inhibitors or Angiopoietin recombinant or Angiopoietin chimeric proteins to the subject.

6. A method for protection and/or improvement of renal function and alleviation of acute kidney injury symptoms in a subject in need thereof, the method comprising administering to the subject a pharmaceutical composition comprising one or more agents capable of TIE2 receptor activation, such as VE-PTP inhibitors or Angiopoietin recombinant or Angiopoietin chimeric proteins to the subject.

7. The method or treatment of any one of claims 1 through 6, wherein the one or more agents capable of TIE2 receptor activation, such as VE-PTP inhibitors or Angiopoietin recombinant or Angiopoietin chimeric proteins are administered to the subject prior to the infliction of acute kidney injury or disease, or ischemia-reperfusion injury; during infliction of acute kidney injury or disease, or ischemia-reperfusion injury; and/or after infliction of acute kidney injury or disease, or ischemia-reperfusion injury.

8. The method or treatment of claim 7, wherein ischemia reperfusion injury occurs in the setting of acute coronary syndrome, acute kidney injury, intestinal ischemia and reperfusion, stroke, sickle cell disease, sleep apnea, major surgery, or solid organ transplantation.

9. The method or treatment of claim 7, wherein inhibition of VE-PTP is used in combination with anti-inflammatory and/or anti-oxidant therapy/ies.

10. The method or treatment of claim 7, wherein the agent(s) activate(s) the Tie2 receptor either directly or by inhibiting its negative regulator VE-PTP, optionally, are selected from the agents described in paragraph [020] of the application.

11. The method or treatment of claim 6, wherein renal/kidney function is measured by any method know by one of ordinary skill in the art (albumin to creatinine ratio, albuminuria, serum urea, inulin clearance, radioisotopic methods, radiocontrasting agents, Cystatin C, and/or glomerular filtration rate and staging of kidney disease).

12. The method or treatment of any one of claim 7, wherein signs and symptoms of kidney injury/failure include too little urine leaving the body, blood in urine, Swelling in legs, ankles, and/or around the eyes, Fatigue or tiredness, Shortness of breath, Seizures or coma in severe cases, confusion, nausea, chest pain or pressure, high blood pressure, dehydradation, drowsiness, hemorrhage, fever, rash, bloody diarrhea, severe vomiting, abdominal pain, pale skin, edema, and/or detectable abdominal mass.

13. The method or treatment of claim 7, wherein kidney injury is caused by infection, dehydration, recent surgery, trauma, exposure to heavy metals or toxic solvents, a condition that obstructs blood flow (e.g., cardia arrest), medications, kidney stones, blood vessel abnormalities that affect blood flow to/from/within the kidney, glomerulonephritis, lupus, blockage in the ureters, low blood pressure, bleeding too much, severe diarrhea, heart disease or heart attack, liver failure, non-steroidal anti-inflammatory drugs (e.g., aspirin, ibuprofen, naproxen), serious burns, severe allergic reaction, blood cloths in or around the kidneys, chemotherapy, antibiotics, contrast dyes used during CT scans, MRI scans, and other imaging tests, alcohol abuse, drug abuse, cancer, enlarged prostate, diabetes, virus infections (e.g., coronavirus).

14. The method or treatment of claim 7, wherein the treatment is combined with hemodialysis, peritoneal dialysis, medicines to control the amounts of vitamins and minerals (e.g., potassium, calcium) in the blood (e.g., calcium, glucose or sodium polystyrene sulfonate (Kionex), treatments to keep the right amount of fluid in the blood (e.g., IV fluids, diuretics), diet, kidney transplant, steroids, acthar, rituximab, cyclophosphamide, mycophenalate mofetil, ACE inhibitors, angiotensin II receptor blockers, cyclosporine, tracrolimus, sirolimus, liposorber LA-15, and combinations thereof.

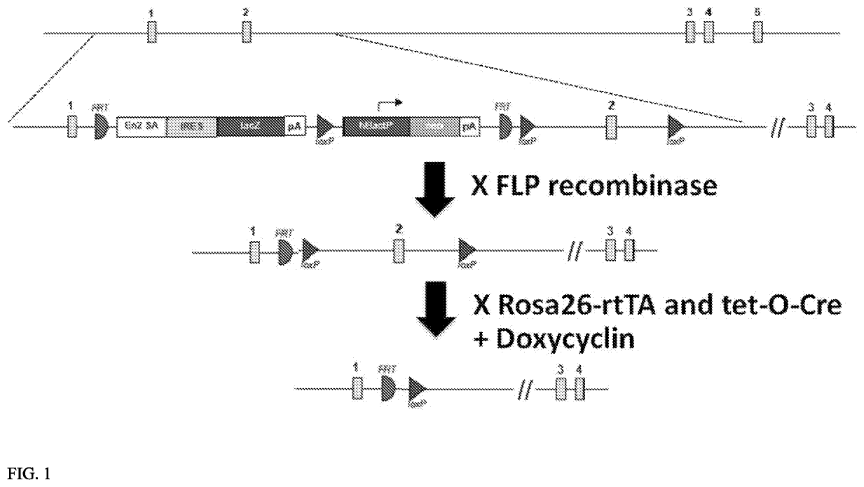

15. A VE-PTP knockout mice produced by a the method that comprises the steps of FIG. 1.

16. A VE-PTP knockout mice wherein a bitransgenic doxycycline-inducible system (Veptp^{flox/flow}, Rosa26-rtTA^{+/+}, tetO-Cre^{Tg/+}) is used to knockout the VE-PTP gene from the vasculature of mice at postnatal day 0 (VE-PTPiKO).
